(19) United States
(12) Patent Application Publication
(10) Pub. No.: US 2020/0163614 A1
(43) Pub. Date: May 28, 2020

(54) AN IMPROVED DEVICE FOR DETECTING, PREVENTING, MONITORING AND TREATING PARAFUNCTIONAL ACTIVITIES IN ODONTOLOGICAL FIELD

(71) Applicant: Lorenzo Noveri, Sanremo (IM) (IT)

(72) Inventor: Lorenzo Noveri, Sanremo (IM) (IT)

(21) Appl. No.: 16/638,421

(22) PCT Filed: Aug. 28, 2017
(86) PCT No.: PCT/IT2017/000182

§ 371 Date: Feb. 11, 2020

(51) Int. Cl. A61B 5/00; A61B 5/0488; H04R 1/46

(52) U.S. Cl. CPC A61B 5/4557; A61B 5/4803; A61B 5/0488; A61B 5/4519; A61B 5/486; H04R 1/46; A61B 5/6833

(57) ABSTRACT

The present invention concerns a device (1) for detecting a parafunctional activity and comprising at least a sensor (3, 103) configured to detect a muscular activity and a processor (110) programmed to recognize a parafunctional activity by means of an analysis of the electromyographic signal detected by the sensor. According to the invention, a device for detecting sounds (150, C) is further comprised, configured to recognize a voice emitted by the user and cooperating with the processor.

### TECHNICAL FIELD

The present invention refers to the technical field concerning medical instruments.

In particular, the invention refers to a device allowing to detect, prevent, monitor and treat parafunctional activities over time, in particular those regarding odontological field.

### BACKGROUND ART

The parafunctional activity is defined as an abnormal, not physiological, muscular activity without functional significance, which occurs between a function and another one, while instead the musculature should rest.

The parafunctional activity may occur both while awake and while asleep.

Almost the entirety of parafunctional patients does not realize their parafunctional activity but only suffers symptoms of damages caused by this disease. Among the various parafunctional activities in odontological field, the most important (as they are more damaging) and frequent ones are bruxism, teeth-grinding and jaw clenching. Under normal conditions, when the mouth does not move and therefore is at rest, mandibular posture is determined by the balance of the muscular tones and at this stage the teeth are not in contact with each other. Physiologically, the teeth of the antagonist arches come into contact with each other only, and not always, while swallowing; this means that, if the antagonist teeth contact each other outside of the swallowing, then a parafunctional activity is occurring. Therefore, the common denominator of these parafunctions is the dental contact outside swallowing. Depending on the type of contact and the mandibular movement, bruxism is distinguished from teeth-grinding and jaw clenching. Parafunctions occur both while asleep and while awake and the damage caused by this disease is directly proportional to their intensity (power) and frequency. The most frequent damages caused by the parafunctional activity concern:
Teeth (teeth wear, thermal sensitivity, fractures, mobility);
Gums (recessions, inflammations, gingival pockets);
Bone (rarefaction, bony pockets);
Muscles (pain near the ear, temple, under the cheekbone, the jaw, neck, back, during chewing, muscle-tense headache, limitation to open and move the mouth);
Joints (joint noises near the ear, articulatory snap while opening and closing the mouth, arthritis and mandibular arthrosis);
Vertigo;
Tinnitus;
Sleep disorders.

At the present time, many electronic devices exist trying to detect a parafunctional activity, in order to emit a signal or input persuading the patient to stop it.

For example, on 27 Nov. 2015 the same inventor of the present PCT application filed an Italian patent application with number UB2015A005983, in which he describes a device that can be applied onto skin, for example behind the ear.

The device includes a sensor (S) able to detect muscular activity and a processor (P) programmed to recognize a normal muscular activity from a parafunctional activity, analyzing the duration time (T) of the detected muscular activity and the time interval between an activity and the subsequent one.

Therefore, in the case of parafunctional activity, it occurs generally according to specific parameters of frequency and duration different from physiological ones.

Other similar devices work according to that principle, for example WO98/31277.

A technical problem is that devices working only by means of an analysis of electromyographic signals, for example in terms of duration and frequency of muscular activity, are not perfectly accurate and functional, especially in categorizing a phenomenon during the waking period.

During the waking period, but also during sleep, although to a lesser extent, the facial mimic activity associated with speech is frequent and continues and its duration, frequency or intensity detected by sensors can lead to mix up such voluntary activity with a parafunctional activity.

While asleep, voice activity is minimized (for example, it is possible to talk involuntarily if you are dreaming) but anyway this event may be mixed up with a parafunctional activity. During the day, this activity is so frequent and random that a correct interpretation of the parafunctional activity becomes very difficult.

### DISCLOSURE OF INVENTION

It is therefore the aim of the present invention to provide an innovative device which solves said technical inconveniences.

In particular, the aim of the present invention is to provide a device for monitoring a parafunctional activity, in particular that related to a user's mouth (of odontological type), effectively and without causing any discomfort both while asleep and while awake.

More particularly, the object of the present invention is to provide a reliable device able to detect the parafunctional actions even during the waking hours where the subject speaks, thus adopting the facial expression.

These and other aims are thus obtained through the present device (1) for detecting a parafunctional activity, according to claim 1.

Such a device (1) comprises sensing means (3, 103) configured to detect a muscular activity and a processor (110) programmed for recognizing a parafunctional activity by means of an analysis of the electromyographic signal detected by said sensing means.

According to the invention, a device for detecting sounds (150, C) is further comprised, cooperating with the processor (100) in such a manner as to distinguish a potential vocal sound emitted in use by the user.

In this way, all said technical inconvenience are easily solved.

Indeed, the device for detecting sounds is able to detect the voice of the subject wearing the device and to send the related signal to the processor.

In particular, the device for detecting sounds, a microphone for example, can only transduce physically all the surrounding sounds (voice, noise, etc.). Analogic band-pass filter being part of the device for detecting sounds limit this signal to a frequency range centered on the human voice. The processor, for example a microcontroller, acquires the filtered signal and then sets with internal parameters (i.e. specific algorithm) whether the sound is compatible with the patient's voice.

In this way, in an event of contextual detection of an electromyographic signal received by the sensing means showing a parafunctional activity and of the recognition of a vocal sound emitted by the user, the processor categorizes such an event as false positive.

Instead, in the case of detection of only an electromyographic signal proving a parafunctional activity without any detection (i.e. without any recognition) of a vocal sound, the processor identifies such an occurrence as parafunctional activity and can memorize the occurrence and possibly activate also the dissuading device (a vibration, for example).

In this way, it is possible to identify reliably and discard all the false positive determined by the facial expression of the user while speaking.

It is also described here a method for detecting a parafunctional activity and comprising the detection of an electromyographic signal by means of sensing means (3, 103) configured to detect a muscular activity and an analysis of said signal through a processor (110).

According to such a method, a phase of vocal recognition of the subject is further comprised by means of a device for detecting sounds (150, C) cooperating with the processor in such a manner that in the event of concomitant detection of an electromyographic signal compatible with a parafunctional activity and of recognition of the vocal sound emitted by the user, the processor categorizes such an occurrence as false positive. In the case of detection of the electromyographic signal compatible with a parafunctional activity without the recognition of a vocal sound emitted by the user, the processor identifies such an occurrence as parafunctional activity.

Further advantages are inferable by other remaining dependent claims.

### BRIEF DESCRIPTION OF DRAWINGS

Further features and advantages of the present device (1; 101), according to the invention, will become apparent from the following description of preferred embodiments thereof, given only by way of non-limiting, indicative example, with reference to the accompanying drawings, wherein:

FIG. 1 shows a schematization of the device 1, according to a first embodiment, which for example can be applied behind the earlobe and lengthened onwards to the tragus and downwards to the angle of the mandible, in such a manner as to detect the muscular activity of the masseter muscle;

FIG. 2 shows a time advancement of the muscular activity;

FIG. 3 shows a block diagram of the present invention;

FIG. 4 shows a wireless communication between the device, object of the invention, and a device detecting such data, for example a mobile telephony device;

FIG. 5 shows a block diagram of the device 101 according to a second embodiment equal to the first except for the addition of a device for detecting sounds (C, 150) cooperating with the processor 110;

FIG. 6 refers to block A depicted in FIG. 5, where the circuitry required to amplify the signal read by the superficial electromyography sensors is depicted; the analogic signal acquired by differential method is amplified and filtered so that it can be digitally converted;

FIG. 7 shows block B depicted in FIG. 5 and particularizes a portion of the circuits, useful to rectify and integrate the signal. The depicted analogical one is useful for making the analysis and the analog/digital conversion of the signal easier. Both blocks A and B are identical to what is included also in the first embodiment of the invention;

FIG. 8 shows a block C always depicted in FIG. 5 and representing the filters for smoothing the signal received by the microphone; such a FIG. 8 depicts the electrical scheme of one of the possible implementations useful to filter the sound acquired by the microphone; the depicted filter can be used to filter the frequencies of the human voice over other surrounding sounds.

FIG. 9 is a schematic layout showing the application of the option with the microphone close to the ear; the double arrows show the case of voice detection together with the detection of muscular activity while speaking and therefore it is decoded as a false positive;

FIG. 10 is an overall flowchart highlighting the operating phases in accordance with this embodiment with microphone.

### DESCRIPTION OF SOME PREFERRED EMBODIMENTS

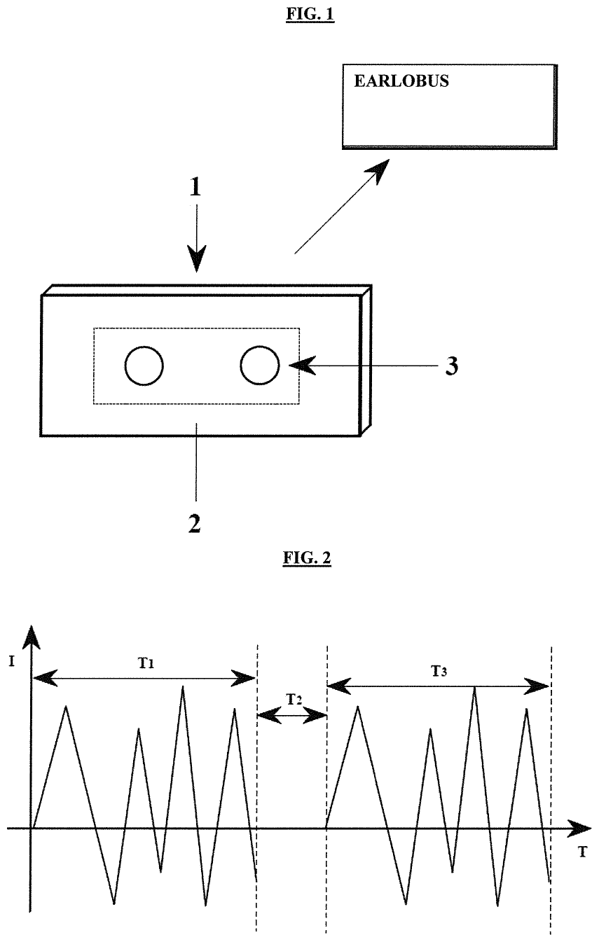

With reference to FIG. 1, according to a first embodiment of the invention, it is described here a device which can be applied onto skin, by means of, for example, a surface or an adhesive support.

The adhesive support 2 can be in the form of a sheet on which the further described element are arranged. Therefore, it is similar to a normal patch.

The adhesive part can be preferably of the interchangeable type and, for example providing an additional double-sided adhesive sheet connected to the sheet forming the support of the element described further.

The overall sizes of the adhesive support can vary depending on needs. Thanks to available technologies, very small sizes can be provided. Therefore, this allows an application onto skin in suitable positions preventing the device from causing disturbances or inconveniences, thus proving not to be invasive and so functioning.

Moreover, as it will be described below, all the components are inserted in said adhesive support, such that no connections to external PCs are needed by means of wirings in general, by making such a device very convenient when using it both while sleeping and while being awake.

Thanks to the adhesive support, such a device can be placed by the patient onto skin either behind, or in front or just below the earlobe, in correspondence of the angle of the mandible and near the skin part of the mandibular insertion of the masseter muscle. Alternatively, it can be placed onto the skin in front and just above the ear by the skin part of the mandibular insertion of the temporal muscle. These positions are convenient, as the sensor can work by detecting the muscular activity of said two muscles, the most used ones during the mouth parafunctional activity.

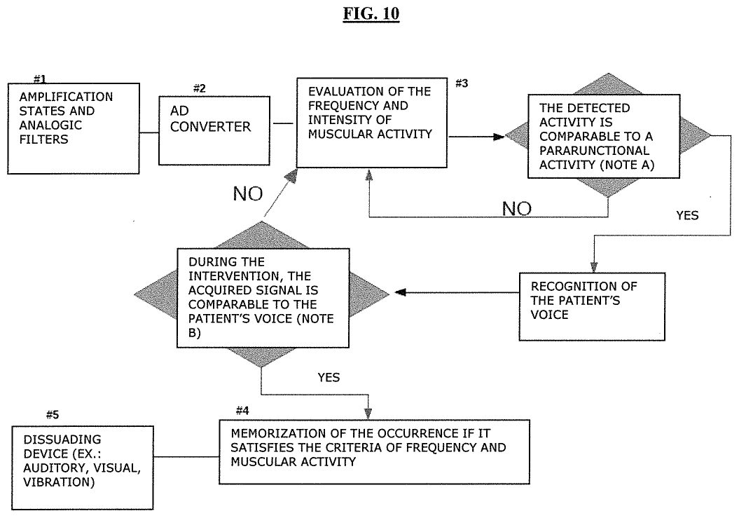

FIG. 9 depicts schematically an example, even if showing the specific case of the second embodiment described below.

Therefore, FIG. 1 outlines the adhesive support 2 and highlights a further area 3.

Such an area 3 includes a system of sensors for detecting muscular activity, for example two sensors of superficial electromyography.

In particular, as commonly used in the field of electromyography, two sensors are preferably provided, i.e. a couple of electrodes. A third auxiliary sensor, or electrode, is generally used for reducing the noise.

Each sensor can be for example an electrode or an electrode array able to receive the electric (electromyographic) signals produced by the contraction of the local musculature affected by the parafunctional activity. Therefore, the device 1 is generally applied to a point on the skin and the detecting system 3 is able to read the corresponding muscular activity in the area where it is applied.

Such sensors are already known in the background and they are used in the field of electromyography, also for different purposes and uses and for this reason they are not described further here.

Alternatively, sensors made by a deformable electric element which modifies its resistance to the electrical current passage according to its shape could be used. Its deformation indicates muscular activity and it can be measured by checking its resistance variation with respect to a standard value determined by its rest condition.

Then a system for ADC conversion is provided inserted in the adhesive support. Indeed, the sensor provides analogic data which must be converted to digital for their subsequent memorization, sending and processing.

Then a memory buffer/processor is provided. Such a processor analyses data sent by the detecting unit. Its task is to recognize voluntary and physiological muscular activity (for example swallowing) from the parafunctional activity, as it is described further.

An actuating device for treating the parafunctional activity is always inserted in such an adhesive support. For this purpose, such a device activates a response signal in the case of a parafunctional activity, for example a short vibration (produced for example by a piezoelectric actuator or a similar one, as for the “vibration” function of a common cellphone). In another embodiment, such an actuating device can inject a short electrical current with such a frequency not to cause pain or ache to the patient.

Then a memory card is provided for memorizing data related to the parafunctional activity.

An antenna allows to transmit memorized data to an external device, in particular to an application (for example on the patient's personal cellphone) which on its turn transmits them to a detecting station.

At last a battery, held in the device itself, is provided for supplying energy to the system and for the functions of sensing, vibration, memorization and sending of wireless data.

A scheme which shows such components arranged on the adhesive support is depicted in FIG. 3.

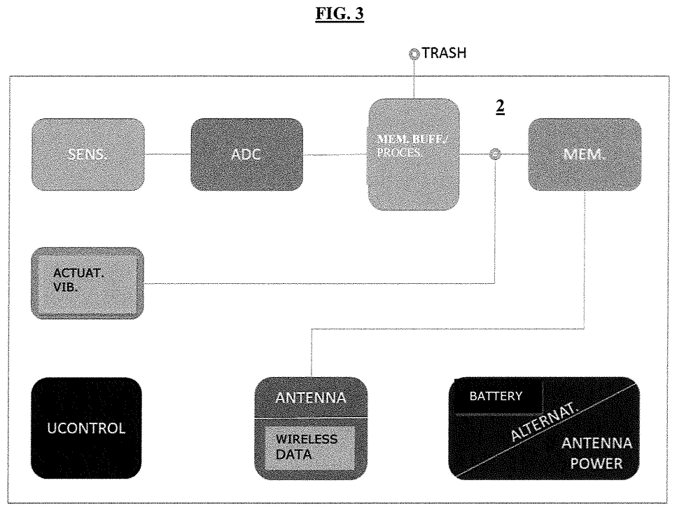

As shown in FIG. 2, the principle for determining an odontological parafunctional activity through the sensing system is based on the determination of electromyographic signals detected by the sensor which prove a parafunctional activity.

In a preferred embodiment of the invention such electromyographic signal can be the duration time (T) of the muscular activity and its frequency. Frequency means here the time interval between an occurrence and another one.

Indeed, the swallowing occurs by activating the musculature for a duration of a second approximately and with a frequency of no more than once a minute. Instead, in the case of parafunctional activity, the musculature is activated for more than a second (i.e. the duration time of muscular activity is more than a second) and with a frequency of less than a minute (i.e. the rest interval between an occurrence and the subsequent one is less than a minute).

Accordingly, the processor receiving the signals of muscular activity can be easily programmed on such bases (i.e. duration and frequency) in such a manner as to recognize a parafunctional activity from a normal function, which is then rejected and not recorded.

Therefore, the variables under monitoring are the duration of the muscular activity (T) and the time interval (LT) when the muscular activity (i.e. frequency) stops. What is considered function is rejected while the parafunctional activity is recorded on a memory, as per scheme of FIG. 3, and allows the activation of the actuator (for example, vibration) for the response.

Therefore, if the detected muscular activity is comprised in predetermined ranges of duration and frequency, this is categorized as parafunctional activity.

A further memory is designated to send data to an external device by means of an antenna, as described above.

Therefore, only for example, FIG. 2 shows a first occurrence detected by the sensor, measuring a series of electric pulses in time T1. This shows the fact that muscle activity, independently of the applied muscular power, is recorded for a certain time T1. Subsequently, the sensor records a pause interval T2 and then begins to record a new T3 duration phenomenon.

As a matter of principle, the indicative ranges of a parafunctional activity are those for which each event has a duration longer than a second: T1>1 sec; and the interval between an occurrence and the next one less than a minute: ΔT<1 min.

Therefore, the processor is programmed to measure these time periods of muscular activity and subsequent pauses between an activity and the next one. If a T1 activity and a T3 activity are detected in a given time interval above a predetermined threshold value with a T2 pause below a predetermined threshold value, then as described, this is recognized as a parafunctional phenomenon which is stored and the actuator is activated at the same time.

The vibration is preferably at 120 Hz since such frequency is known to be used for stretch musculature and therefore it is suitable for such purposes.

In a further embodiment of the invention, such electromyographic signals that are analyzed for the determination of a parafunctional activity concern the duration of the occurrence and the muscular intensity (I).

Therefore, if the duration and muscle intensity fall within predetermined values for parafunctional activity (programmed values and known to the processor exactly as in the case above), the processor identifies this event as a parafunctional activity.

In a further embodiment of the invention, the processor could be programmed to detect parafuntional activity by analyzing only one or a combination of said parameters between duration, frequency and intensity of muscular activity.

Obviously, any electromyographic signal indicative or useful for determining a parafunctional activity can be generally used.

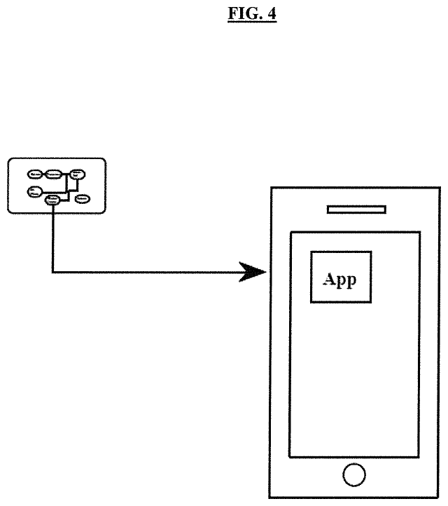

As described, the system can easily include, as shown in FIG. 4, wireless communication with an external device, for example through an application (APP) on the patient's cellphone and which in turn allows to store data.

In this way, the user normally logs in the application and the system uploads such data to the application.

Then a medical center may have access to such data to monitor patients.

As mentioned, all the components are of small size, such as a coin, and can be applied to an adhesive support. However, the support can also not be adhesive and connect with different support systems, such as a small belt to tie around the neck.

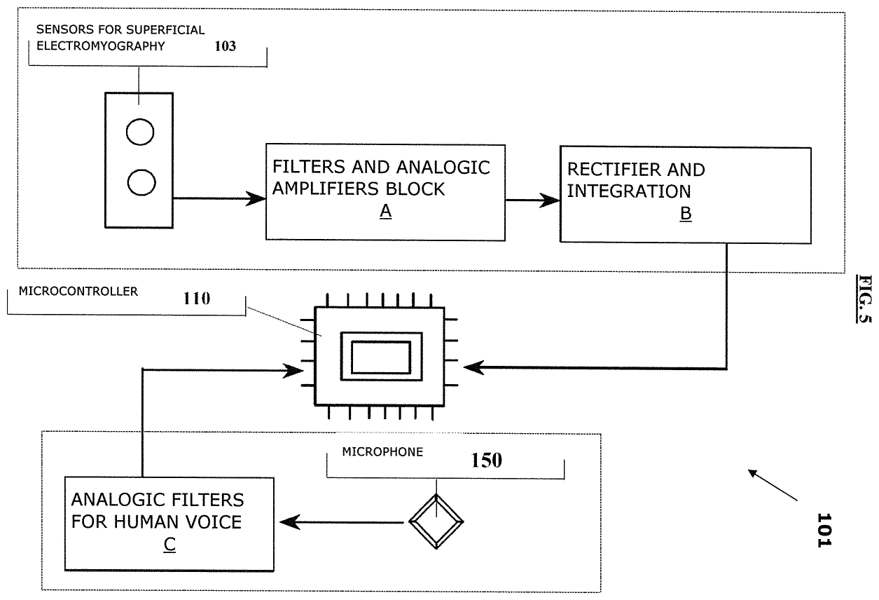

Subject to what has been described above, a preferred embodiment of the invention is described with reference to subsequent FIGS. 5 to 10.

This variant applies everything described above, in particular in terms of electromyographic detection to detect indicative parafunctional muscular activity as well as the provided components.

Therefore, in this case, subject to what has been described above, the new embodiment adds a microphone 150 in order to exceed predetermined functional limits due to “false positive” determined by vocal activity.

As introduced in the field of technical inconveniences, it is known that vocal activity (i.e. speaking) may cause an error (a false positive), as this activity determines muscular activity detected by electromyographic sensor which mey be included in the parameters identifying a parafunctional activity. Depending on the length of the vocal action, the muscular activity linked to it may likely be included in said parameters of duration, frequency and/or muscular intensity with a real risk of being mixed up with parafunctional activity, as it is actually a false positive.

Indeed, above all during daytime activities and waking hours, the subject may speak and therefore have such a muscular activity that could be related to a frequency and/or duration and/or intensity related to the parafunctional activity.

Indeed, during the speech, the sensors detect muscular activity, which obviously must be rejected.

The insertion of the microphone, as explained below, solves this technical inconvenience by allowing it to check whether voice recognition is associated with the detection of muscular activity at the same time.

FIG. 5 shows an overall outline according to this solution.

As previously described, the whole can be arranged on a support, for example adhesive, applicable for example near the ear lobe, as shown in the diagram of FIG. 1, or on a support of a different type.

This embodiment is configured on the support in the following manner:

The processor 110 is represented in the form of a microcontroller and obviously it is also in the first embodiment. The processor manages the various operations and recognizes whether the received information can be classified as a parafunctional activity exactly as in the previously described embodiments and therefore according to the above-mentioned muscular frequency and/or duration of the muscular occurrence.

As explained further, the microcontroller adds to this function the voice recognition that includes the use of a voice recognition algorithm based on a possible signal received by the microphone.

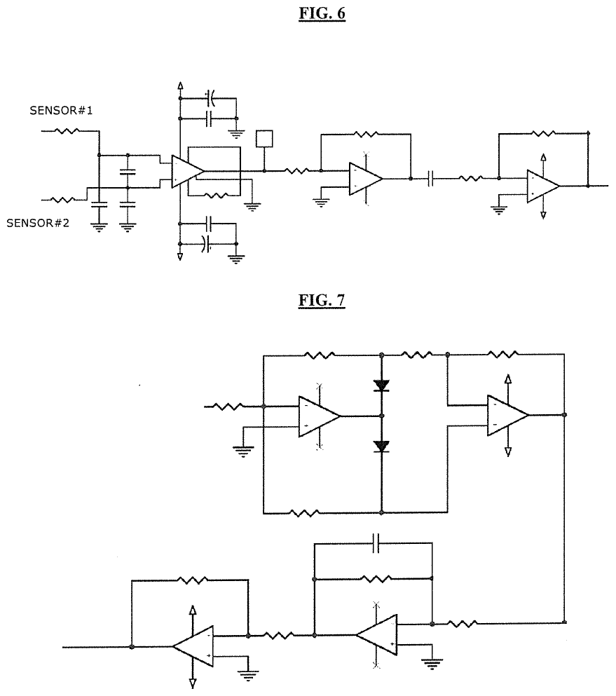

FIG. 5 shows two sensors of electromyographic surface 103 to which the electronic components are connected, represented in blocks (A) and (B) of FIGS. 6 and 7, being also on the first embodiment. They refer to various filters and rectifier which, as per background art, make the signal smooth and readable by the microcontroller.

According to the schematization of FIG. 5, the microphone 150 is provided for acquiring sounds emitted in the surroundings.

In the preferred embodiment of the invention, the microphone is comprised in the support itself and then its suitable size is like a pinhead.

Nowadays many suitable types of microphones with such sizes exist. The chosen useful technology has the purpose of an easy insertion of the microphone into the device both for reducing its sizes and encumbrance and for guaranteeing at the same time enough perception to easily detect the patient's voice.

For that purpose, the device includes preferably a microphone of the MME type or other future technology for allowing both small sizes and enough perception to detect the patient's voice.

A possible embodiment of the invention including a throat microphone or an external microphone is not excluded (for example by comprising it in an external device and not in the same support).

Obviously, the embodiment comprising the microphone in the same adhesive support allows much smaller sizes.

The filters (block C of FIG. 5) arranged downstream the microphone allow to cancel background noises.

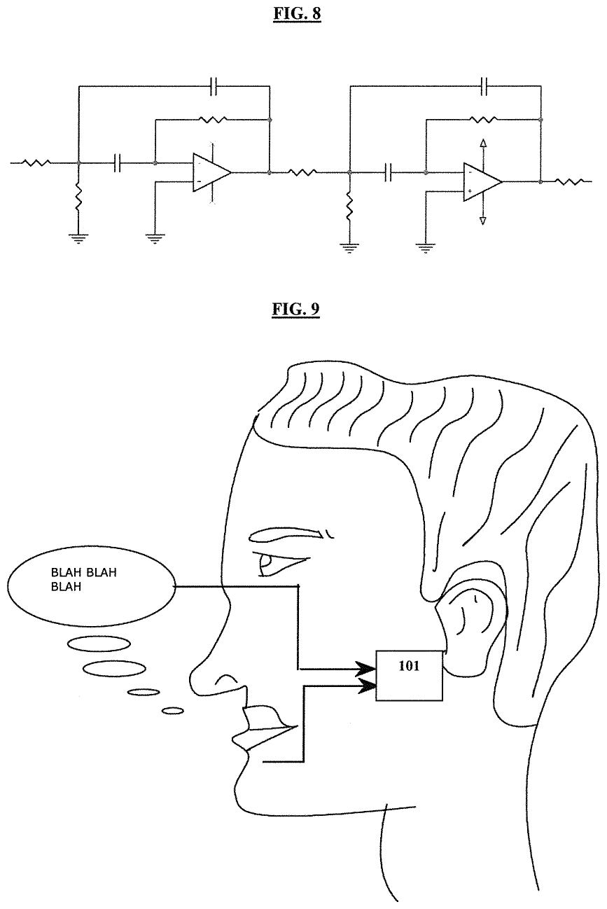

Such filters are described in detail in FIG. 8 and are shown as block C in FIG. 5. They are able to smooth the background noise and so to transmit only the user's voice to the microprocessor, if the voice is emitted.

In particular, the embodiment is as follows: microphone is physically sensitive to every noise. Then filters and amplifiers “smooth” the signal from most part of the background noise. Then the microprocessor, with suitably arranged algorithms, analyzes the signal to decide whether the received signal is compatible with the sound emitted by the patient.

Therefore, if no voice is emitted by the user but there is background noise, such a filter c does not transmit any information for the microcontroller or anyway then the received information is rejected by the processor thanks to its algorithm of recognition of the patient's voice.

The filter, as per scheme of FIG. 8, is based on band-pass filters focused on the distinctive frequency interval of human voice.

Obviously, with reference to FIG. 5, the dissuading device and the potential system for communicating with the external software can be included in the support, exactly as described in the previous embodiments.

The device comprises an initial calibration phase to modulate levels depending on the signal/noise ratio of the contact and to evaluate the subjective signal level of the patient.

Basically, an initial calibration by emitting a vocal sound by the user determines an indicative reference. The device is based not only on the information acquired during the calibration process but also compares the signal with internal models to categorize whether muscular activity is comparable to that generated by a parafunctional activity.

At this point, as shown by the flowchart of FIG. 10, the operation is as follows:

When there is muscle activity, the sensor (or sensors) detects that activity which is sent to microcontroller 110, as shown in FIG. 5.

The microcontroller must check whether this muscular activity falls into a parafunctional activity case by verifying, with reference to such muscular activity, the frequency and/or duration and/or muscular intensity. If detected frequency and/or duration and/or muscular intensity, as mentioned above, fall into predetermined preset ranges, then this is potentially recognized as a parafunctional activity.

This is shown in the first part of the flowchart marked with the steps (#1, #2 and #3) until it arrives at the verification phase if the detected activity is comparable to a parafunctional activity. If it is not, a new muscle activity has to be analyzed and therefore the micro-controller does not carry out any operation.

However, in the case that the electromyographic signals detected through the sensors are recognized by the microcontroller as a possible parafunctional event, then the analysis of the signal received by the microphone occurs. In this case, the direction of the arrow indicated by YES in the flowchart leads to the voice recognition (“Patient's Voice Recognition” box).

In that sense, by means of the microphone, a potential parafunctional activity is likely to be only “speaking”, whether the user's voice has been detected by muscular activity.

The provided filters, as mentioned, cancel background noise, so if the microphone detects a sound, this is probably the voice of the patient.

The processor analyzes the filtered signal by the microphone with a special algorithm to categorize it as a voice or not.

Therefore, if this sound is recognized as the patient's vocal sound, then the microprocessor simultaneously receives both an electromyographic signal from the sensors compatible with a parafunctional activity and a signal by the microphones downstream the microphone, compatible as voice. It classifies this information as a false positive. Indeed, the possible signal of parafunctional activity is liked to a muscular activity of speaking.

However, if, according to the electromyographic signal indicating a parafunctional activity, the microcontroller does not receive any signal by the microphone or receives a signal categorized as a background noise, then this information is categorized as a parafunctional activity.

Therefore, if the microcontroller does not receive any signal filtered by the above-mentioned filters by the microphone that is compatible with the patient's voice then the detected activity is classified as a parafunctional activity, if it falls within the frequency and duration parameters.

Then this occurrence is stored with the subsequent actuation of the dissuading device.

Otherwise, if the microcontroller receives a signal, filtered from the above-described filters, by the microphone that is compatible with the voice of the patient, and at the same time the activity received by the sensors falls between the parafunctional activity, then this event is rejected as a false positive.

Ultimately, always with reference to the flowchart of FIG. 10:

Sensors to check muscular activity are always operating.

When the device measures muscular activity over certain criteria (such as frequency and intensity as mentioned), then the device enters “DETECTION” mode (interval of a few seconds).

In this mode, in addition to muscular activity, the microphone signal is also acquired.

In this “DETECTION” range, it is set whether the activity is comparable to a parafunctional activity. In particular, if a microprocessor receives a sound signal by the microphone and its filters, then this is analyzed and if the sound is recognized as voice, referring to the flowchart of FIG. 10, the NO line is followed, leading to a new occurrence.

If no voice is detected, then the YES line that leads to the storing and the implementation of the dissuading device is followed.

The microcontroller, therefore, does not always analyze the voice (or more generally the surrounding sound), but analyzes and acquires the voice only when necessary.

The electromyographic signals for detecting a parafunctional activity are those cited in the description and any other known ones used for a long time to verify a parafunctional activity and they are not a specific object of the present invention. For this reason, they are not described here further.

Finally, in a further embodiment of the invention, subject to what has been described above, the present device can be associated with a further stress detection device, for example in the form of a wearable bracelet.

The bracelet, similarly to the described device, can include sensors suitable for detecting body stress, together with the same filters and A/D singnal converters.

Both devices can be communicating with a data recording device, such as the App provided in the mobile device. In this manner, once stored, a whole monitoring can be examined by specialized personnel. Such bodily stress indicators related with the detected occurrences of parafunctional activities can be useful in determining a correct case history of the subject and for example identifying a correct therapy, as well as providing statistical correlation data between parafunctional activity and stress states.

1-16. (canceled)

17. A device for detecting a parafunctional activity and comprising:
sensing means configured to detect an electromyographic signal produced by a muscular activity;
processor programmed for recognizing a parafunctional activity by means of an analysis of the said electromyographic signal;
a detecting sounds device cooperating with the processor in such a manner as to distinguish a potential vocal sound emitted in use by the user;
a dissuading device for preventing the parafunctional activity;
characterized in that the processor and the detecting sounds device cooperate with each other in such a manner that, in the event of concomitant detection of an electromyographic signal compatible with a parafunctional activity and of recognition of the vocal sound emitted by the user, the processor categorizes such an occurrence as false positive and in the case of detection of the electromyographic signal compatible with a parafunctional activity without the recognition of a vocal sound emitted by the user, the processor identifies such an occurrence as parafunctional activity, said device being in form of an adhesive support, able to be applied to the user's face, on which said sensing means, the processor and the detecting sounds device are arranged and wherein, further, said dissuading device is inserted in said adhesive support and is activated in response to the identification of said parafunctional activity in the said case of detection of the electromyographic signal compatible with a parafunctional activity without the recognition of a vocal sound emitted by the user.

18. The device according to claim 17, wherein the detecting sounds device comprises at choice:
a microphone;
a throat microphone.

19. The device according to claim 18, wherein the microphone is of the MEMS type.

20. The device according to claim 17, wherein the detecting sounds device further comprises an analogic filter for the human voice, wherein the analogic filter is an analogic band-pass filter.

21. The device according to claim 17, wherein the processor is a microcontroller.

22. The device according to claim 17, wherein a block of filters and analogical amplification and a block of rectifier and integration are further comprised for signals coming from the sensor upstream the processor.

23. The device according to claim 17, wherein the sensing means are in the form of a sensor of surface electromyography.

24. The device according to claim 17, wherein two or more than two sensors of surface electromyography are provided.

25. The device according to claim 17, wherein the processor is programmed to check if an electromyographic signal is indicating a parafunctional activity on the basis of the frequency between an occurrence of a signal and the subsequent one and the duration of each signal.

26. The device according to claim 17, wherein the processor is programmed to check if an electromyographic signal is indicating a parafunctional activity on the basis of the frequency between an occurrence of a signal and the subsequent one and the intensity of each signal.

27. The device according to claim 17, wherein the processor is programmed to check if an electromyographic signal is indicating a parafunctional activity on the basis of the analysis of at least one out of the following elements:
frequency between an occurrence of a signal and the subsequent one;
intensity of the signal;
duration of each signal.

28. The device according to claim 17, wherein the dissuading device is a vibrating device able to cause a vibration in the subject.

29. An assembly including:
a first device detecting a parafunctional activity and comprising:
sensing means configured to detect an electromyographic signal produced by a muscular activity;
processor programmed for recognizing a parafunctional activity by means of an analysis of the said electromyographic signal;
a detecting sounds device cooperating with the processor in such a manner as to distinguish a potential vocal sound emitted in use by the user;
a dissuading device for preventing the parafunctional activity;
characterized in that the processor and the detecting sounds device cooperate with each other in such a manner that, in the event of concomitant detection of an electromyographic signal compatible with a parafunctional activity and of recognition of the vocal sound emitted by the user, the processor categorizes such an occurrence as false positive and in the case of detection of the electromyographic signal compatible with a parafunctional activity without the recognition of a vocal sound emitted by the user, the processor identifies such an occurrence as parafunctional activity, said device being in form of an adhesive support, able to be applied to the user's face, on which said sensing means, the processor and the detecting sounds device are arranged and wherein, further, said dissuading device is inserted in said adhesive support and is activated in response to the identification of said parafunctional activity in the said case of detection of the electromyographic signal compatible with a parafunctional activity without the recognition of a vocal sound emitted by the user;
a second device comprising one or more sensors for detecting the status of body stress.

30. A method for detecting a parafunctional activity, comprising the phase of detection of an electromyographic signal by means of sensing means configured to detect a muscular activity and an analysis of said signal through a processor and wherein a phase of vocal recognition of the subject is further comprised by means of a detecting sounds device cooperating with the processor in such a manner that in the event of concomitant detection of an electromyographic signal compatible with a parafunctional activity and of recognition of the vocal sound emitted by the user, the processor categorizes such an occurrence as false positive and the case of detection of the electromyographic signal compatible with a parafunctional activity without the recognition of a vocal sound emitted by the user, the processor identifies such an occurrence as parafunctional activity.

31. The method according to claim 30, wherein in the case of parafunctional activity the actuation of a dissuading device is provided.
